(19) United States
(12) Patent Application Publication
(10) Pub. No.: US 2004/0210205 A1
(43) Pub. Date: Oct. 21, 2004

(54) Disposable pant-like undergarment

(75) Inventors: Paul VanGompel, Hortonville, WI (US); Jacqueline Ann Gross, Neenah, WI (US); Russell Evan Thorson, Appleton, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/414,986

(22) Filed: Apr. 15, 2003

(51) Int. Cl. A61F 13/15; A61F 13/20

(52) U.S. Cl. 604/385.22

(57) ABSTRACT

A disposable pant-like undergarment is disclosed which has a stretchable front panel and a stretchable back panel. An absorbent assembly having a liquid pervious bodyside liner, a liquid-impervious outer cover, and an absorbent positioned therebetween is secured to an outer surface of the front and back panels. The bodyside liner is partially overlapped by the front and back panels such that a physical barrier is established between a portion of the bodyside liner and the user's skin. The absorbent assembly is folded to enable the first and second panels to overlap one another and a pair of seams are formed which join the front and back panels together to create a pant-like undergarment having a waist opening and a pair of leg openings.

### BACKGROUND OF THE INVENTION

A disposable pant-like undergarment is designed for absorbing human exudate. The pant-like undergarment is similar in appearance, size and shape to a regular cloth underwear except that it is not designed to be laundered and reused two or more times. A disposable pant-like undergarment is intended to be worn by persons, including infants, toddlers, or adults, and is designed for single or temporary use. The disposable pant-like undergarment is meant to be disposed of after being used once instead of being laundered or dry cleaned for re-use. The disposable pant-like undergarment is designed to be pulled up around the user's torso without having to first open the undergarment in order to place it on a person's body. The stretchablity of the material used to construct the disposable pant-like undergarment permits the undergarment to snugly conform to the anatomy of the user's torso. The disposable pant-like undergarment can be manufactured to be an infant diaper, a child training pant, an adult incontinence garment, a feminine menstrual pant, etc.

Some disposable pant-like undergarments manufactured today resemble regular cloth underwear in that they have a waist opening and a pair of leg openings. Such disposable pant-like undergarments can be pulled up around the torso of a user in a similar fashion as regular cloth underwear. It has been found that a disposable, absorbent pant-like undergarment is generally more discreet than a refastenable undergarment because it does not contain the extra material forming the hook or adhesive fasteners and loop or adhesive landing zone materials. A disposable pant-like undergarment tends to be sleeker, smoother and slimmer especially around the waist opening. For this reason, many adults suffering from incontinence tend to prefer the pant-like design. Today, many disposable pant-like undergarments utilize an absorbent assembly that is secured to the inner or bodyside surface of the front and back panels so as to be in direct contact with the user's body. One disadvantage with this design is that as the undergarment fills with body fluid, such as urine, or with solid human excrement, the absorbent assembly has limited ability to expand outward. This means that as the absorbent assembly gets insulted with urine, the wet body fluid is maintained adjacent to the user's body and can create an uncomfortable wet feel. With solid human excrement, the current undergarments tend to hold the waste material in direct contact with the skin of the user. This feature can create an uncomfortable feeling for the user.

Now a disposable pant-like undergarment for absorbing human body fluid and solid excrement has been invented that allows the absorbent assembly to expand outward so as to provide a dryer and more comfortable feel to the user.

### SUMMARY OF THE INVENTION

Briefly, this invention relates to a disposable pant-like undergarment for absorbing human body fluid and solid excrement. The pant-like undergarment includes a stretchable front panel and a stretchable back panel, each having an inner surface, an outer surface, and first and second side edges. The pant-like undergarment also includes an absorbent assembly having a liquid pervious bodyside liner, a liquid-impervious outer cover, and an absorbent positioned therebetween. The absorbent assembly has a first end and a second end. The first end of the absorbent assembly is secured to the outer surface of the front panel and the second end of the absorbent assembly is secured to the outer surface of the back panel. The bodyside liner is partially overlapped by the front and back panels such that a physical barrier is established between a portion of the bodyside liner and a user's skin. The absorbent assembly is capable of being folded to enable the first and second side edges of the front panel to align with the first and second side edges of the back panel. The front and back panels are secured together at the first and second side edges by a pair of seams to form a pant-like undergarment having a waist opening and a pair of leg openings.

### BRIEF DESCRIPTION OF THE DRAWINGS

FIG. 1 is a perspective view of a disposable pant-like undergarment.

FIG. 2 is a plane view of a disposable pant-like undergarment having an absorbent assembly secured to the outer surface of the stretchable front and back panels and showing a partial cut away view of the construction of the absorbent assembly.

FIG. 3 is a side view of the disposable pant-like undergarment shown in FIG. 2.

FIG. 4 is a cross-sectional view of the disposable pant-like undergarment shown in FIG. 2 taken along line 4-4.

FIG. 5 is a cross-sectional view of the disposable pant-like undergarment shown in FIG. 2 taken along line 5-5.

### DETAILED DESCRIPTION

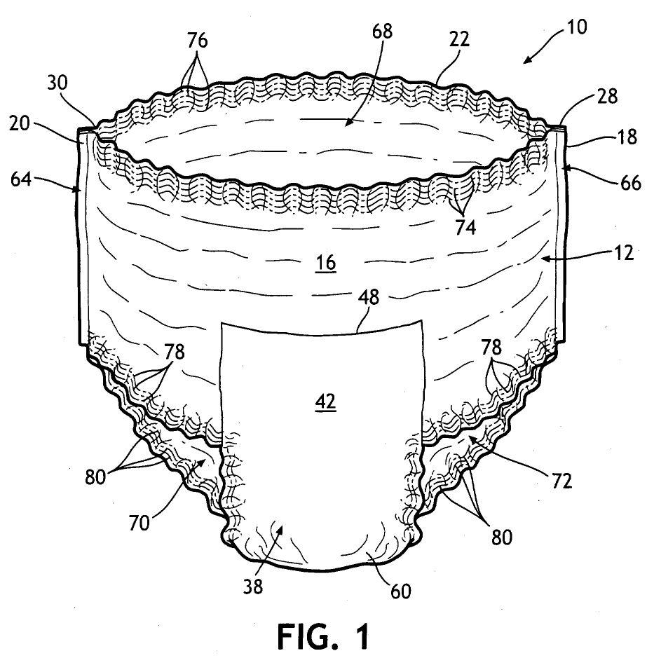

Referring to FIG. 1, a disposable pant-like absorbent undergarment 10 is depicted. The disposable pant-like undergarment 10 is intended to be worn by persons, including infants, toddlers, or adults, and is designed for a single or temporary use. The disposable pant-like undergarment 10 is meant to be disposed of after being used once instead of being laundered or dry cleaned for re-use. The disposable pant-like undergarment 10 is designed to be pulled up around the user's torso without having to first open the undergarment 10 in order to place it on a person's body. In FIG. 1, the undergarment 10 is shown as it would appear just prior to being pulled up around a user's torso.

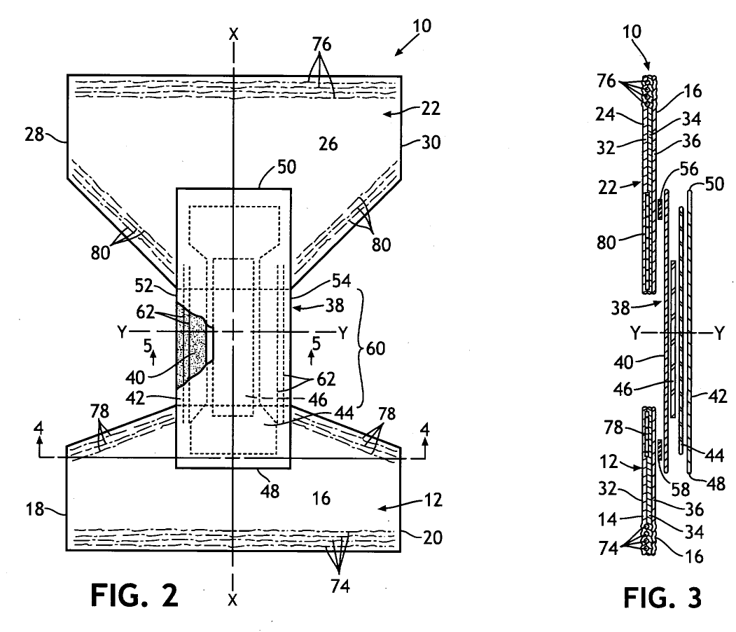

Referring to FIGS. 2 and 3, the disposable pant-like undergarment 10 is depicted in an open position solely for the purpose of better showing the various components. The open undergarment has a longitudinal central axis X-X and a transverse central axis Y-Y. The pant-like undergarment 10 includes a stretchable front panel 12 having an inner surface 14, an outer surface 16, a first side edge 18 and a second side edge 20. The undergarment 10 also includes a stretchable back panel 22 having an inner surface 24, an outer surface 26, a first side edge 28 and a second side edge 30. The front and back panels, 12 and 22 respectively, are discontinuous from one another. The inner surfaces 14 and 24 are in direct contact with the user's skin and are also sometimes referred to as the bodyside surface. The outer surfaces 16 and 26 are situated opposite to the inner surfaces 14 and 24 and are spaced away from the user's skin. The outer surfaces 16 and 26 are also sometimes referred to as the garment facing surfaces since they can be in direct contact with the inner surfaces of the user's outer clothing.

The front and back panels, 12 and 22 respectively, are formed from a stretchable, elastic material or from a stretchable laminate that contains two or more layers wherein at least one of the layers is elastic. By “layer” it is meant a single elastic sheet, an elastic film, an elastic net like material or a plurality of elastic strands. When a plurality of elastic strands is utilized, they can be positioned between two or more outer layers. Each of the front and back panels, 12 and 22 respectively, is formed such that the entire panel 12 and 22 is capable of being stretched or elongated and is also capable of being contracted or restricted. By “entire” is meant that approximately 100% of the front panel 12 is stretchable and approximately 100% of the back panel 22 is stretchable.

Referring to FIG. 3, each of the front and back panels, 12 and 22 respectively, are shown being formed as a three layer laminate. Each of the front and back panels, 12 and 22 respectively, includes a first layer 32, a second or middle layer 34, and a third layer 36. The first and third layers, 32 and 36 respectively, are the outer layers and can be formed from the same material or from different materials. The first and third layers, 32 and 36 respectively, can be constructed from natural or synthetic fibers and can be a woven or non-woven material. The second or middle layer 34 has the elastic properties and is sandwiched between the first and third layers, 32 and 36 respectively. It should be noted that one or both of the outer layers 32 or 36 can be made from an elastic material, if desired.

The elastic laminate provides stretch or elongation, and contraction or restriction in at least one direction. For the purpose of this invention, “stretch or stretchable” shall be defined as to lengthen, widen, or distend by applying a force, such as by pulling. “Elongation” shall be defined as the act of elongating or the condition of being elongated, including an extension; to make or grow longer, lengthen, extend, or to make slender. For example, elongation can refer to a ratio determined by measuring the difference between the initial unextended length of a material and its extended length in a particular dimension and dividing that difference by the initial unextended length of the material in that same dimension. This value is then multiplied by 100 when one wishes to express elongation as a percent. “Contraction” shall be defined as the act of contracting or the state of being contracted or shortened; to reduce in size or length, to shrink or to shorten. For example, a 1 inch (2.54 cm) strip of material can be stretched to about 2 inches (about 5 cm) and then when the stretching force is removed, the material will return towards or to its original 1 inch (2.54 cm) length. “Restriction” shall be defined as the act of restricting or the state of being restricted; keeping within limits, confinement.

The stretch, elongation, contraction or restriction can be in one direction or in two or more directions. Desirably, the stretch, elongation, contraction or restriction of the front and back panels, 12 and 22 respectively, are in a direction approximately parallel to the transverse axis Y-Y. The transverse direction extends laterally across the torso from one hip bone to the other hip bone. More desirably, the stretch, elongation, contraction or restriction of the front and back panels, 12 and 22 respectively, are in at least two directions, one direction being approximately parallel to the longitudinal axis X-X and the other approximately parallel to the transverse axis Y-Y. Most desirably, the stretch, elongation, contraction or restriction of the front and back panels 12 and 22 are in multiple directions, or stated another way, in three or more directions extending over an arc of 360 degrees. The ability of the front and back panels, 12 and 22 respectively, to contract will provide a restrictive force during use of the disposable pant-like undergarment 10 to ensure that it snugly conforms to the anatomy of the wearer's torso.

Desirably, the front and back panels, 12 and 22 respectively, are constructed from a stretch bonded laminate (SBL) where the elastic core or middle layer 34 is elongated before the two outer nonwoven layers 32 and 36 are attached. The attachment can be by an adhesive, by heat, by pressure, by a combination of heat and pressure, etc. Another material option for the front and back panels, 12 and 22 respectively, is a necked bonded laminate (NBL). The NBL material is also a three-layer laminate but the elastic core or middle layer 34 is not pre-stretched prior to being attached to the two outer nonwoven layers 32 and 36. The outer layers 32 and 36 are necked stretched before the elastic core or middle layer 34 is attached to them.

It should also be noted that the front and back panels, 12 and 22 respectively, can be constructed from an elastic film that is capable of being stretched in at least one direction and desirably in both the machine direction and the cross-direction. Alternatively, the front and back panels, 12 and 22 respectively, can be an elastic nonwoven that has a machine direction stretch or a cross-direction stretch. Extensible materials can also be used to form the front and back panels, 12 and 22 respectively, of the disposable pant-like undergarment 10.

Alternatively, the front and back panels, 12 and 22 respectively, can be formed from two outer layers 32 and 36 with a plurality of elastic strands sandwiched therebetween. The elastic strands can be formed from LYCRA®. LYCRA® is a registered trademark of E. I. Du Pont De Nemours & Co., having an office at 1007 Market Street, Wilmington, Del. 19898. The elastic strands can be aligned approximately parallel to one another or be angled or skewed relative to one another. The elastic strands can also be uniformly or randomly spaced apart from one another. The elastic strands can vary in shape, size, configuration, and/or length. The diameter and/or cross-sectional configuration of the elastic strands, the decitex (weight in grams per 10,000 meters of a strand) of the elastic strands, and the tension imparted into the elastic strands can all be varied to suit one's particular product needs. The elastic strands can have a round, semi-circular, square, rectangular, oval or some other geometrical configuration. The elastic strands can overlap, intersect or crisscross at least one other elastic strand. The various ways of positioning, orienting, and adhering the elastic strands to the two outer layers 32 and 36 are well known to those skilled in the art.

Referring again to FIGS. 1 and 2, the disposable pant-like undergarment 10 also includes an absorbent assembly 38. The absorbent assembly 38 includes a liquid pervious bodyside liner 40, a liquid-impervious outer cover 42, and an absorbent 44 positioned therebetween. A surge layer 46 can be optionally used, which is located between the bodyside liner 40 and the absorbent 44. The surge layer 46 can function to rapidly acquire and temporarily retain body fluid, such as urine, before it can be absorbed into the absorbent 44. Desirably, the surge layer 46 is also capable of wicking the body fluid lengthwise and/or widthwise across its surface as well as directing the body fluid downward in a z direction toward the absorbent 44.

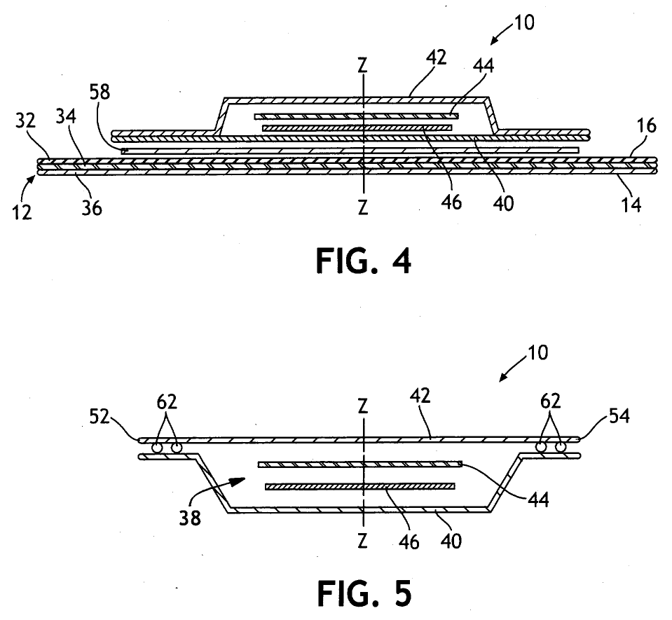

Referring to FIGS. 2, 3 and 4, the absorbent assembly 38 has a first end 48, a second end 50, a first side edge 52 and a second side edge 54. The absorbent assembly 38 is secured to the outer surface 16 of the front panel 12 approximate the first end 48 by an attachment 56 and is secured to the outer surface 26 of the back panel 22 approximate the second end 50 by an attachment 58. The absorbent assembly 38 is secured to the front and back panels, 12 and 22 respectively, after each panel has been stretched a predetermined amount. By attaching the absorbent assembly 38 to the outer surfaces 16 and 26 of the front and back panels, 12 and 22 respectively, the absorbent assembly 38 is capable of expanding outward away from the user's torso while the front and back panels, 12 and 22 respectively, maintain their snug position against the user's torso. This unique ability for the absorbent assembly 38 to freely expand outward away from the user's torso without restrictions from the elastic front and back panels, 12 and 22 respectively, produces a more functional undergarment. The absorbent assembly 38 is capable of taking in and retaining additional body fluid as it expands outward, more so than if it was restricted by being secured to the inside surfaces 14 and 24 of the front and back panels, 12 and 22 respectively.

The attachments 56 and 58 can be by various means and can include permanent attachments as well as removable or releasable attachments. Desirably, the attachments 56 and 58 are permanent attachments where they are not designed to be removed without destroying the bond. The attachments 56 and 58 can be formed by using glue, adhesive, ultrasonic bonds, heat bonds, pressure bonds, heat and pressure bonds, etc. The attachments 56 and 58 can also include a mechanical fastener, such as by sewing with thread, using buttons and button holes, using snaps, by employing hook and loop fasteners, etc. A hook and loop fastener is generally considered a releasable attachment. One type of hook and loop fastener is VELCRO® wherein a hook material is releasably engaged into a loop material. VELCRO® is a registered trademark of Velcro USA, Inc. having an office at 406 Brown Avenue, Manchester, N.H. 03103.

The attachments 56 and 58 can be formed along a continuous line or over a surface area having a predetermined length and width. Alternatively, the attachments 56 and 58 can consist of intermittent point bonds that are spaced apart from one another. For example, the intermittent point bonds can be formed by using a hot or cold melt adhesive or by forming ultrasonic bonds. Various bond formations can be used which are known to those skilled in the art. Desirably, the attachments 56 and 58 are formed using intermittent bonds because it allows the elastic material forming the front and back panels, 12 and 22 respectively, to gather the absorbent assembly 38 as the elastic contracts. This gathering feature causes the absorbent assembly 38 to move outward away from the user's body prior to being insulted with body fluid.

The front and back panels, 12 and 22 respectively, can be stretched in a direction approximately parallel to the transverse axis Y-Y, or in any other direction or directions, before the absorbent assembly 38 is secured to it. The amount of stretch can vary. The front and back panels, 12 and 22 respectively, can be stretched at least about 15% from a relaxed state before the absorbent assembly 38 is secured thereto. Desirably, the front and back panels, 12 and 22 respectively, can be stretched at least about 25% from a relaxed state before the absorbent assembly 38 is secured thereto. More desirably, the front and back panels, 12 and 22 respectively, can be stretched at least about 50% from a relaxed state before the absorbent assembly 38 is secured thereto. Most desirably, the front and back panels, 12 and 22 respectively, can be stretched from between about 55% to about 1,000% from a relaxed state before the absorbent assembly 38 is secured thereto. The front and back panels, 12 and 22 respectively, extend laterally beyond the first and second side edges 52 and 54 of the absorbent assembly 38. The front panel 12 also extends longitudinally beyond the first end 48 of the absorbent assembly 38 and the back panel 22 extends longitudinally beyond the second end 50 of the absorbent assembly 38. This lateral and longitudinal extension allows the front and back panels, 12 and 22 respectively, to elastically conform to the body of the wearer.

Referring now to FIGS. 2 and 5, when the absorbent assembly 38 is secured to the front and back panels, 12 and 22 respectively, a crotch region 60 is formed. The crotch region 60 separates the front panel 12 from the back panel 22 and is designed to cover the perineum area of the wearer. The crotch region 60 can cover a distance of a few inches in an infant diaper to several inches in an adult incontinence garment. For example, a crotch region 60 in an infant diaper may range from about 2 inches (about 5 centimeters(cm)) to about 10 inches (about 25 cm). In an adult incontinence garment; the crotch region 60 may range from about 6 inches (about 15 cm) to about 20 inches (about 51 cm).

In FIG. 2, one can clearly sees that the bodyside liner 40 of the absorbent assembly 38 is partially overlapped by both the front panel 12 and the back panel 22. This overlap establishes a physical barrier between a portion of the bodyside liner 40 and the user's skin. The material of the front and back panels, 12 and 22 respectively, that overlap the bodyside liner 40 does not have to be permanently secured in place to the absorbent assembly 38 but does serve as an extra barrier layer to prevent rewet. “Rewet” is a condition where as the absorbent assembly 38 is compressed by movement of the user, for example by having the user sit down, body fluid retained by the absorbent 44 may be forced back towards the bodyside liner 40. As the body fluid contacts the bodyside liner 40, the user can experience a wet uncomfortable feeling. By having less of the bodyside liner 40 in direct contact with the user's skin, the user is less likely to experience this wet feeling.

The portions of the front and back panels, 12 and 22 respectively, which overlap the bodyside liner 40 can vary from between about 10% to about 50% of the surface area of the bodyside liner 40. Desirably, the front and back panels, 12 and 22 respectively, will overlap at least about 15% of the bodyside liner 40. More desirably, the front and back panels, 12 and 22 respectively, will overlap at least about 20% of the bodyside liner 40. Most desirably, the front and back panels, 12 and 22 respectively, will overlap at least about 25% of the bodyside liner 40.

The absorbent assembly 38 can be stretchable or non-stretchable in relation to the front and back panels, 12 and 22 respectively. Desirably, the absorbent assembly 38 is non-stretchable in relation to the front and back panels, 12 and 22 respectively. By having the absorbent assembly 38 be non-stretchable in relation to the front and back panels, 12 and 22 respectively, it is meant that the absorbent assembly 38 will not stretch appreciably in the longitudinal or transverse directions. The reason for this is that the front and back panels, 12 and 22 respectively, are elastically stretchable and can contract to snugly conform to the user's anatomy, especially to his or her torso. Alternatively, the absorbent assembly 38 could be constructed with a pleated or folded construction, so as to be capable of being expanded in the longitudinal or transverse directions, if expansion of the absorbent assembly is needed. The pleating or folding should occur before the absorbent assembly 38 is secured to the front and back panels, 12 and 22 respectively.

Normally, there is no need to have the absorbent assembly 38 gather as the front and back panels, 12 and 22 respectively, contract. However, the absorbent assembly 38 can be constructed and attached to the front and back panels, 12 and 22 respectively, in a way that will allow the absorbent assembly 38 to be gathered as the front and back panels, 12 and 22 respectively, elastically contract in the transverse direction. In either circumstance, the absorbent assembly 38 should remain over the perineum. As the absorbent assembly 38 receives body fluid and/or excrement discharged by the wearer, it will be displaced outward, away from the user's torso. The ability of the absorbent assembly 38 to move outward away from the user's torso as additional body fluid is absorbed and retained is a direct result of the fact that it is secured to the outer surfaces 16 and 26 of the front and back panels, 12 and 22 respectively. The attachments 56 and 58 assure that the absorbent assembly 38 covers the perineum but is capable of moving outward away from the torso as additional body fluid is received and retained.

Referring now to FIGS. 2 and 5, the disposable pant-like undergarment 10 also has at least one elastic member 62 positioned adjacent to and aligned approximately parallel to each of the first and second side edges 52 and 54 of the absorbent assembly 38. Each of the elastic members 62 is situated between the bodyside liner 40 and the outer cover 42. The elastic members 62 provide a gasket to hold the edges 52 and 54 of the absorbent assembly 38 against the user's body. Each of the elastic members 62 can be in the form of an elastic strand, ribbon or strip. Desirably, from about two to six elastic members 62 will be positioned adjacent to each of the side edges 52 and 54. In FIGS. 2 and 5, two elastic members 62 are shown positioned adjacent to each of the side edges 52 and 54. The elastic members 62 are shown as having a round cross-sectional configuration although various other geometrical configurations can be utilized. The elastic members 62 extend parallel to the longitudinal axis X-X and extend completely through the crotch region 60. Desirably, the ends of the elastic members 62 will be located within the front and back panels, 12 and 22 respectively.

Referring again to FIGS. 1 and 2, the absorbent assembly 38 is capable of being folded transversely, approximate the transverse axis Y-Y, to enable the first and second side edges 18 and 20 of the front panel 12 to align with the first and second side edges 28 and 30 of the back panel 22, respectively. A pair of seams 64 and 66 is then formed to join the front panel 12 to the back panel 22. The seam 64 secures the second side edge 20 of the front panel 12 to the second side edge 30 of the back panel 22 while the seam 66 secures the first side edge 18 of the front panel 12 to the first side edge 28 of the back panel 22. One will notice that FIG. 2 is a view of the exterior or outer surface of the undergarment 10 and the front panel 12 has to be folded into the page in order to keep the absorbent assembly 38 on the outside of the undergarment 10. The folded undergarment 10 then has to be rotated 180 degrees to arrive at the view shown in FIG. 1. After the pair of seams 64 and 66 is formed, the pant-like undergarment 10 has a waist opening 68 and a pair of leg openings 70 and 72. Since the front and back panels, 12 and 22 respectively, are formed from a stretchable elastic material, the waist opening 68 and the pair of leg openings 70 and 72 can expand or contract in size to accommodate the anatomy of the user.

The disposable pant-like absorbent undergarment 10 can also include one or more elastic strands of waist elastics 74 and 76 positioned in the front and back panels, 12 and 22 respectively, approximate the waist opening 68. Four elastic strands 74 and 76 are depicted in the drawings, although it should be understood that from 1 to about 25 elastic strands 74 and 76 could be used, if desired. The elastic strands 74 and 76 can be positioned between the first and second layers, 32 and 34 respectively, as shown. Alternatively, the elastic strands 74 and 76 can be positioned between the second and third layers, 34 and 36 respectively, if desired. Normally, the elastic strands 74 and 76 are aligned approximately parallel to the transverse axis Y-Y and extend across each of the front and back panels, 12 and 22 respectively. The elastic strands 74 and 76 can be uniformly or randomly spaced apart from one another and are normally located within about 2 inches (about 5.4 cm) of the waist opening 68.

Lastly, the disposable pant-like absorbent undergarment 10 can also include one or more elastic strands of leg elastics 78 and 80 positioned in the front and back panels, 12 and 22 respectively, approximate the leg openings 70 and 72. Three elastic strands 78 and 80 are depicted in the drawings, although it should be understood that from 1 to about 10 elastic strands 78 and 80 could be used, if desired. The elastic strands 78 and 80 can be positioned between the first and second layers, 32 and 34 respectively, as shown in FIG. 2. Alternatively, the elastic strands 78 and 80 can be positioned between the second and third layers, 34 and 36 respectively, if desired. Normally, the elastic strands 78 and 80 are aligned approximately parallel to the edges of the front and back panels, 12 and 22 respectively, that are situated adjacent to the absorbent assembly 38. The elastic strands 78 and 80 can be uniformly or randomly spaced apart from one another and are normally located within about 0.3 inches (about 0.76 cm) of the leg opening 70 and 72.

While the invention has been described in conjunction with a specific embodiment, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A disposable pant-like undergarment comprising:
a) a stretchable front panel having an outer surface, and first and second side edges;
b) a stretchable back panel having an outer surface, and first and second side edges;
c) an absorbent assembly including a liquid pervious bodyside liner, a liquid-impervious outer cover, and an absorbent positioned therebetween, said absorbent assembly having a first end and a second end, said absorbent assembly being secured to said outer surface of said front panel approximate said first end and being secured to said outer surface of said back panel approximate said second end, said bodyside liner being partially overlapped by said front and back panels such that a physical barrier is established between a portion of said bodyside liner and a user's skin, and said absorbent assembly capable of being folded to enable said first and second side edges of said front panel to align with said first and second side edges of said back panel; and
d) a pair of seams joining said front and back panels together at said first and second side edges to form a pant-like undergarment having a waist opening and a pair of leg openings.

2. The disposable pant-like undergarment of claim 1 wherein said undergarment has a longitudinal axis and a transverse axis and said entire front panel is stretchable in a direction that is approximately parallel to said transverse axis.

3. The disposable pant-like undergarment of claim 1 wherein said undergarment has a longitudinal axis and a transverse axis and said entire back panel is stretchable in a direction that is approximately parallel to said transverse axis.

4. The disposable pant-like undergarment of claim 1 wherein said front and back panels are stretched in a direction approximately parallel to said transverse axis before said absorbent assembly is secured thereto.

5. The disposable pant-like undergarment of claim 4 wherein said front and back panels are stretched at least about 15% from a relaxed state before said absorbent assembly is secured thereto.

6. The disposable pant-like undergarment of claim 1 wherein said absorbent assembly is bonded at intermittent points along said first and second ends to said front and back panels.

7. The disposable pant-like undergarment of claim 6 wherein said intermittent points are ultrasonically bonded.

8. The disposable pant-like undergarment of claim 6 wherein said intermittent points are adhesively bonded.

9. The disposable pant-like undergarment of claim 1 wherein each of said front and back panels is stretchable in at least two directions, one direction being approximately parallel to said longitudinal axis and a second direction being approximately parallel to said transverse axis.

10. A disposable pant-like undergarment comprising:
a) an entirely stretchable front panel having an outer surface, and first and second side edges;
b) an entirely stretchable back panel having an outer surface, and first and second side edges;
c) an absorbent assembly including a liquid pervious bodyside liner, a liquid-impervious outer cover, and an absorbent positioned therebetween, said absorbent assembly having a first end, a second end, a first side edge and a second side edge, said absorbent assembly being secured to said outer surface of said front panel approximate said first end and being secured to said outer surface of said back panel approximate said second end to form a crotch region, said bodyside liner being partially overlapped by said front and back panels such that a physical barrier is established between a portion of said bodyside liner and a user's skin, said absorbent assembly being non-stretchable in relation to said front and back panels, and said absorbent assembly capable of being folded to enable said first and second side edges of said front panel to align with said first and second side edges, respectively, of said back panel;
d) at least one elastic member positioned adjacent to each of said first and second side edges of said absorbent assembly and situated between said bodyside liner and said outer cover; and
e) a pair of seams joining said front and back panels together at said first and second side edges to form a pant-like undergarment having a waist opening and a pair of leg openings.

11. The disposable pant-like undergarment of claim 10 wherein said undergarment has a longitudinal axis and a transverse axis and said front and back panels are stretched in a direction that is approximately parallel to said transverse axis before said absorbent assembly is secured thereto.

12. The disposable pant-like undergarment of claim 11 wherein said front and back panels are stretched at least about 50% from a relaxed state before said absorbent assembly is secured thereto.

13. The disposable pant-like undergarment of claim 10 wherein said front and back panels overlap at least about 15% of said bodyside liner.

14. The disposable pant-like undergarment of claim 13 wherein said front and back panels overlap at least about 25% of said bodyside liner.

15. The disposable pant-like undergarment of claim 10 wherein as said front and back panels snugly conform to a user's torso, said absorbent assembly is gathered and displaced outward away from said user's torso.

16. A disposable pant-like undergarment comprising:
a) an entirely stretchable, elastic front panel having an outer surface, and first and second side edges;
b) an entirely stretchable, elastic back panel having an outer surface, and first and second side edges;
c) an absorbent assembly including a liquid pervious bodyside liner, a liquid-impervious outer cover, and an absorbent positioned therebetween, said absorbent assembly having a first end, a second end, a first side edge and a second side edge, said absorbent assembly being secured to said outer surface of said front panel approximate said first end and being secured to said outer surface of said back panel approximate said second end, said bodyside liner being partially overlapped by said front and back panels such that a physical barrier is established between a portion of said bodyside liner and a user's skin, said absorbent assembly being non-stretchable in relation to said front and back panels, and said absorbent assembly capable of being folded to enable said first and second side edges of said front panel to align with said first and second side edges, respectively, of said back panel;
d) at least one elastic member positioned adjacent to and aligned approximately parallel to each of said first and second side edges of said absorbent assembly and situated between said bodyside liner and said outer cover; and
e) a pair of seams joining said front and back panels together at said first and second side edges to form a pant-like undergarment having a waist opening and a pair of leg openings.

17. The disposable pant-like undergarment of claim 16 wherein said front and back panels overlap at least about 15% of said bodyside liner.

18. The disposable pant-like undergarment of claim 17 wherein said front and back panels overlap at least about 20% of said bodyside liner.

19. The disposable pant-like undergarment of claim 18 wherein said front and back panels overlap at least about 25% of said bodyside liner.

20. The disposable pant-like undergarment of claim 16 wherein as said front and back panels elastically contract to snugly conform to a user's torso, said absorbent assembly is gathered and displaced outward away from said user's torso.
